(19) United States
(12) Patent Application Publication
(10) Pub. No.: US 2015/0158024 A1
(43) Pub. Date: Jun. 11, 2015

(54) DEHYDROGENATION CATALYST FOR HYDROCARBONS AND METHOD OF PREPARATION THEREOF

(71) Applicant: Reliance Industries Limited, Mumbai (IN)

(72) Inventors: Sharad Vasudeorao Lande, Akola (IN); Veera Venkata Satya Bhaskara Sita Rama Murthy Katravulapalli, Hyderabad (IN); Sreedharan Unnikrishnan, Vadodara (IN); Nagesh Sharma, Vadodara (IN); Shashank Vaidya, Thane (IN); Raksh Vir Jasra, Vadodara (IN)

(21) Appl. No.: 14/621,792

(22) Filed: Feb. 13, 2015

Related U.S. Application Data
(63) Continuation of application No. PCT/IN2013/000435, filed on Jul. 15, 2013.

(30) Foreign Application Priority Data
Aug. 13, 2012 (IN) 1716/MUM/2012

(51) Int. Cl. B01J 37/18; B01J 21/04; B01J 37/02; B01J 27/135; B01J 37/00; B01J 35/10; B01J 35/02; B01J 23/02; C07C 5/333; B01J 37/08

(52) U.S. Cl. CPC B01J 37/18; C07C 5/3337; B01J 21/04; B01J 37/0242; B01J 37/08; B01J 37/0018; B01J 35/1014; B01J 35/026; B01J 35/1019; B01J 23/02; B01J 27/135; C07C 2521/04; C07C 2523/62

(57) ABSTRACT

The present disclosure relates to a dehydrogenation catalyst composite comprising at least one alumina support impregnated with at least one layer of at least one alkaline earth metal element and at least one layer comprising at least one catalytic metal element, at least one group VIA element and optionally, at least one halogen element. The present disclosure also relates to a process for preparation of the dehydrogenation catalyst composite.

### CROSS REFERENCE TO RELATED APPLICATIONS

This application is a continuation application of International Application PCT/IN2013/000435, filed on Jul. 15, 2013, which claims the benefit of Indian Patent Application No. 1716/MUM/2012, filed on Aug. 13, 2012. All of these applications are incorporated by reference in their entirety.

### FIELD OF DISCLOSURE

The present disclosure relates to a catalyst composite and a process for its preparation. Particularly, the present disclosure relates to a dehydrogenation catalyst composite and a process for its preparation.

### BACKGROUND

Dehydrogenation of saturated hydrocarbons or paraffins, specifically C₂-C₂₀ paraffins, is an important petrochemical process because of the increasing demand for unsaturated hydrocarbons. These unsaturated hydrocarbons are olefinic monomers, such as ethylene, propylene, butenes, butadiene, styrene and straight chain mono olefins of carbon number ranging from C₆-C₂₀, which find extensive applications in the production of a variety of plastics, synthetic rubber and detergents. Furthermore, dehydrogenation of naphthenes and paraffins are important reactions during catalytic reforming processes practiced worldwide for the production of aromatics (BTX) and high octane gasoline.

Platinum and platinum-containing bimetallic catalysts supported on alumina are widely used for heavy linear paraffins dehydrogenation in the petrochemical industry. However, it is observed that these dehydrogenation catalysts undergo rapid deactivation, mainly due to fouling by heavy carbonaceous materials.

U.S. Pat. No. 4,786,625 discloses a novel catalytic composite comprising a platinum group metal element; a modifier metal element selected from the group consisting of tin, germanium, rhenium and mixtures thereof; an optional alkali or alkaline earth metal element or mixtures thereof, an optional halogen element, and an optional catalytic modifier element on a refractory oxide support having a nominal diameter of at least about 850 microns. The distribution of the surface-impregnated platinum metal element is such that the catalyst has particular utility as a hydrocarbon dehydrogenation catalyst in a hydrocarbon dehydrogenation process.

U.S. Pat. No. 4,812,597 discloses, a dehydrogenation catalyst comprising a modified iron catalyst for a dehydrogenation reaction in which the hydrocarbons such as ethyl benzene are treated with the catalyst. A selective oxidation catalyst, which is also employed, comprises a noble metal of group VIII of the Periodic Table, a metal of group IVA and, if so desired, a metal of Group IA or IIA composited on a porous inorganic support such as alumina.

U.S. Pat. No. 5,358,920 discloses a dehydrogenating catalyst for saturated hydrocarbons comprising platinum, tin, sodium and .tau.-alumina. The support of the catalyst is a large pore diameter .tau.-Al.sub.2 O.sub.3 with dual pore diameter distribution. At least 40% of the total pore volume is contributed by pores with a pore diameter in the range of 1000-10000.

U.S. Pat. No. 4,672,146 discloses a catalyst composite comprising a group VIII, noble metal element, a co-formed IVA metal element, an alkali metal or alkaline earth metal element and an alumina support having a surface area in the range of 5 to 150 m²/g.

U.S. Pat. No. 4,762,960 discloses a novel catalytic composite comprising a platinum group metal element; a modifier metal element selected from the group consisting of tin, germanium, rhenium and mixtures thereof; an alkali or alkaline earth metal or mixtures thereof, an optional halogen element, and an optional catalytic modifier element on a refractory oxide support having a nominal diameter of at least about 850 microns.

U.S. Pat. No. 6,177,381 discloses a layered catalyst composition, a process for preparing the composition and processes for using the composition. The catalyst composition comprises an inner core such as alpha-alumina, and an outer layer bonded to the inner core composed of an outer refractory inorganic oxide such as gamma-alumina. The outer layer is uniformly dispersed on a platinum group metal such as platinum and a promoter metal such as tin. The composition also contains a modifier metal such as lithium.

All the aforesaid catalysts get deactivated primarily because of coke formation which further results in reduced stability, activity and selectivity of the catalyst. Use of alumina as a support material for the dehydrogenation catalysts also accelerates the process of coke formation.

Therefore, there is felt a need for developing a novel dehydrogenation catalyst which not only reduces coke formation but also makes it easy to remove during the dehydrogenation process resulting in improved activity, stability and better dispersion of metal elements.

### OBJECTS

Some of the objects of the present disclosure, which at least one embodiment is able to achieve, are discussed herein below.

It is an object of the present disclosure to provide a novel dehydrogenation catalyst composite.

It is another object of the present disclosure to provide a dehydrogenation catalyst composite having better metal dispersion.

It is yet another object of the present disclosure to provide a dehydrogenation catalyst composite with increased catalytic stability.

It is still another object of the present disclosure to provide a process for the preparation of a dehydrogenation catalyst composite.

It is a further object of the present disclosure to provide a process for the preparation of a dehydrogenation catalyst composite which is safe and economical.

It is still a further object of the present disclosure to ameliorate one or more problems of the prior art or to at least provide a useful alternative.

Other objects and advantages of the present disclosure will be more apparent from the following description which is not intended to limit the scope of the present disclosure.

### BRIEF DESCRIPTION OF THE ACCOMPANYING DRAWING

FIG. 1: illustrates the XRD Patterns for dehydrogenation catalyst of the present disclosure.

### SUMMARY

In accordance with one aspect of the present disclosure there is provided a dehydrogenation catalyst composite comprising:
a. at least one alumina support comprising:
i. a core of alpha alumina; and
ii. at least one layer of alumina selected from the group consisting of gamma alumina, delta alumina and theta alumina surrounding said core,
b. at least one layer comprising at least one alkaline earth metal element selected from the group consisting of magnesium, calcium, barium and strontium impregnated on the surface of said alumina support; and
c. at least one layer comprising:
i. at least one catalytic metal element selected from the group consisting of group VIII elements, group IVA elements, and alkali metal elements;
ii. at least one group VIA element; and
iii. optionally, at least one halogen element,

said layer provided on alkaline earth metal impregnated alumina support.

Typically, the dehydrogenation catalyst of the present disclosure has been characterized by the percentage dispersion of catalytic metal element is in the range of 55% to 80%.

Typically, the dehydrogenation catalyst further comprises at least one binder provided within at least one layer of alumina and/or as a discrete layer between the core and the layer of alumina surrounding the core.

Typically, the binder is at least one polar compound selected from the group consisting water, alcohol and ester, preferably water.

Typically, the average diameter of the alumina support is in the range of 1.8 mm to 2.00 mm and the surface area is in the range of 10 m²/g to 200 m²/g.

Typically, the amount of alkaline earth metal element impregnated on the alumina support is in the range of 1% to 10% with respect to the total mass of the dehydrogenation catalyst composite.

Typically, the group VIII element is at least one selected from the group consisting of platinum, nickel and palladium.

Typically, the group IVA element is at least one selected from the group consisting of tin, and germanium.

Typically, the alkali metal element is at least one selected from the group consisting of sodium, lithium, potassium and cesium.

Typically, the halogen element is at least one selected from the group consisting of chlorine, bromine, fluorine and iodine.

Typically, the amount of group VIII elements ranges between 0.01 and 5%, the amount of group IVA elements ranges between 0.01 and 15%, the amount of alkali metal element ranges between 0.01 and 2% and the amount of halogen element ranges between 0.05 and 0.5%; wherein said amount of each element is based on the total mass of the dehydrogenation catalyst.

Typically, the group VIA element is at least one selected from the group consisting of sulfur, selenium and tellurium, preferably sulfur.

Typically, the amount of group VIA element ranges between 0.01% and 15% with respect to the total mass of the dehydrogenation catalyst.

In accordance with another aspect of the present disclosure there is provided a process for the preparing a dehydrogenation catalyst composite, said process comprising the following steps:
a. preparing an alumina support; said method step of preparing an alumina support comprises the following steps:
I. obtaining a core of alpha alumina;
II. coating the core with a mixture comprising activated alumina and at least one binder to obtain a coated core;
III. hydrating the coated core to obtain hydrated core; and
IV. calcining the hydrated core at a temperature of 800 to 900° C. in presence of air to obtain an alumina support with at least one layer of at least one alumina selected from the group consisting of gamma alumina, delta alumina and theta alumina,
b. impregnating the alumina support with at least one alkaline earth metal compound followed by drying and calcining at a temperature of 500° C. to 700° C. for a time period ranging between 1 to 10 hours to obtain an alumina support impregnated with at least one alkaline earth metal element;
c. impregnating the alumina support impregnated with at least one alkaline earth metal element with a mixture comprising at least one catalytic metal element compound, at least one group VIA element compound and optionally, at least one halogen element compound to obtain a catalyst composite; wherein the catalytic metal element compound is at least one selected from the group consisting of group VIII element compounds, group IVA element compounds and alkali metal element compounds;
d. drying and calcining the catalyst composite to obtain a calcined catalyst composite impregnated with at least one catalytic metal element and at least one group VIA element and
e. contacting the calcined catalyst composite with a stream of hydrogen gas under reducing conditions to obtain a dehydrogenation catalyst composite.

Typically, the binder is at least one polar solvent selected from the group consisting of water, alcohol and ester, preferably water.

Typically, the process for the preparing a dehydrogenation catalyst composite, further comprises the following steps:
a) purging a stream of inert gas at a temperature of 300° C. to 500° C. at a high gas hourly space velocity of 100 to 10000 per hour on the dehydrogenation catalyst composite; and
b) cooling the stream to obtain a blanketed dehydrogenation catalyst composite.

Typically, the surface area of the alumina support is maintained in the range of 10 m²/g to 200 m²/g.

Typically, the alkaline earth metal compound is at least one selected from the group consisting of magnesium nitrate, magnesium acetate, calcium nitrate, barium nitrate and strontium nitrate.

Typically, the alkaline earth metal element is at least one selected from the group consisting of magnesium, calcium, barium and strontium.

Typically, the amount of alkaline earth metal element impregnated on the alumina support is in the range of 1% to 10% with respect to the total mass of the dehydrogenation catalyst composite.

Typically, the group VIII element is at least one selected from the group consisting of platinum, nickel and palladium.

Typically, the group VIII element compound is at least one selected from the group consisting of chloroplatinic acid, palladium nitrate and nickel nitrate.

Typically, the group IVA element is at least one selected from the group consisting of tin and germanium.

Typically, the group IVA element compound is at least one selected from the group consisting of stannous chloride and germanium chloride.

Typically, the alkali metal is at least one selected from the group consisting of sodium, lithium, potassium and cesium.

Typically, the alkali metal compound is at least one selected from the group consisting of, sodium chloride, lithium nitrate, potassium chloride and cesium nitrate.

Typically, the halogen element is at least one selected from the group consisting of chlorine, bromine, fluorine and iodine.

Typically, the halogen element compound is at least one selected from the group consisting of hydrochloric acid, carbon tetrachloride, hydrogen bromide, hydrogen fluoride and hydrogen iodide.

Typically, the amount of group VIII elements ranges between 0.01 and 5%, the amount of alkali metal ranges between 0.01 and 2% and the amount of halogen element ranges between 0.05 and 0.5%; wherein said amount of each element is based on the total mass of the dehydrogenation catalyst composite.

Typically, the group VIA element compound is at least one selected from the group consisting of thioglycolic acid thiomalic acid, selenium sulfide and tellurium tetrachloride.

Typically, the group VIA element is at least one selected from the group consisting of sulfur, selenium and tellurium, preferably sulfur and the amount of group VIA element ranges between 0.01% and 15% with respect to the total mass of the dehydrogenation catalyst.

Typically, the hydrogen gas is maintained at a temperature of 400 to 500° C. for a time period of 4 to 8 hrs.

In accordance with yet another aspect of the present disclosure there is provided a process for the preparation of unsaturated hydrocarbons; said process comprising the following steps:
a) preparing a dehydrogenation catalyst composite as per the process of the present disclosure; and
b) contacting said dehydrogenation catalyst composite with at least one hydrocarbon feed at a temperature ranging between 400° C. and 800° C., at a pressure ranging between 0.1 and 10 atm. and at a liquid hourly space velocity in the range of 0.1 to 100/hr. to obtain unsaturated hydrocarbons.

Typically, the hydrocarbon feed comprises at least one hydrocarbon selected from the group consisting of C2 to C20 hydrocarbons.

Other aspects and advantages will be apparent from the following description and the appended claims.

### DETAILED DESCRIPTION

Dehydrogenation catalysts disclosed in the prior art typically comprise an alumina support impregnated with a group VIII element such as platinum, iridium, osmium, ruthenium, palladium, rhodium along with a group IVA element which includes gallium, tin, lead dispersed either on the shell or throughout the support structure in varying amounts. Further, these catalysts also comprise promoters which include sodium, lithium, potassium and cesium.

Dehydrogenation of saturated hydrocarbons using such catalysts however produce gases, heavy alkylate and aromatics. These get deposited on the catalyst support as well as on metal and get polymerized to form coke. As a result, the catalyst activity goes down gradually due to the build-up of coke.

Most of the prior art uses dehydrogenation catalysts containing alumina as a support mainly due to its capability to bind with metal elements, for achieving high dehydrogenation activity. But strong acidic properties of alumina cause side reactions which are responsible for the coke formation.

Therefore, the inventors of the present disclosure have developed a novel dehydrogenation catalyst composite which comprises an alumina support impregnated with at least one layer comprising at least one alkaline earth metal element which may include magnesium, calcium, barium and strontium and at least one layer comprising at least one catalytic metal element and at least one group VIA element. The impregnation of alumina support with alkaline earth metals blocks the acidic sites of the alumina support and promotes hydrogen spillover which in turn reduces coke formation and also increases the stability of the dehydrogenation catalytic composite of the present disclosure.

Further, the dehydrogenation catalyst composite comprising alkaline earth metal impregnated alumina support inhibits the mobility of the catalytic metal element.

Furthermore, the group VIA element of the present disclosure increases the percent dispersion of the catalytic metal element on the surface of the alkaline earth metal impregnated alumina support and thereby increases the dehydrogenation capacity of the dehydrogenation catalyst.

In accordance with one aspect of the present disclosure there is provided a dehydrogenation catalyst composite which comprises an alumina support impregnated with at least one layer comprising at least one alkaline earth metal and at least one layer comprising at least one catalytic metal element, at least one group VIA element and optionally, at least one halogen element. The dehydrogenation catalyst composite of the present disclosure has been characterized by the 55% to 80% percentage dispersion of catalytic metal element.

The alumina support of the present disclosure comprise an inner core as alpha alumina and an outer layer comprising at least one form of alumina selected from the group consisting of gamma alumina, delta alumina and theta alumina.

In accordance with one embodiment of the present disclosure the binder is provided within at least one layer of alumina.

In accordance with another embodiment of the present disclosure the binder is provided as a discrete layer between the core and the layer of alumina surrounding the core.

The average diameter of the alumina support may be in the range of 1.8 mm to 2.00 mm and the surface area may be in the range of 10 m²/g to 200 m²/g.

The alkaline earth metal may be at least one selected from the group consisting of magnesium, calcium, barium and strontium. The amount of alkaline earth metal element impregnated on the alumina support is in the range of 1% to 10% with respect to the total mass of the dehydrogenation catalyst composite.

The alkaline earth metal may be magnesium and the amount of magnesium may be maintained in the range of 1 to 10% with respect to the total mass of the dehydrogenation catalyst composite of the present disclosure.

The catalytic metal elements may be at least one selected from the group consisting of VIII elements, group IVA elements, alkali metal elements in the range of 0.01 to 5%, 0.01 to 15%, 0.01 to 2%, and 0.01 to 2% respectively with respect to the total mass of the dehydrogenation catalyst composite.

The group VIII element may be at least one selected from the group consisting of platinum, nickel and palladium.

The group IVA element may be at least one selected from the group consisting of tin, and germanium.

The alkali metal may be at least one selected from the group consisting of sodium, lithium, potassium and cesium.

The group VIA element of the present disclosure is a capping agent which may include sulfur, selenium and tellurium and the amount of the group VIA element ranges between 0.01% and 15% with respect to the total mass of the dehydrogenation catalyst composite.

In accordance with one of the embodiment of the present disclosure the group VIA element is sulfur.

In accordance with one of the embodiments of the present disclosure the alkaline earth metal impregnated support may further comprises at least one halogen element selected from the group consisting of chlorine, bromine, fluorine and iodine and the amount of halogen element is maintained in the range of 0.05 to 0.5% with respect to the total mass of the dehydrogenation composite.

In accordance with the second aspect of the present disclosure, there is provided a process for the preparation of a dehydrogenation catalyst composite. The process comprises the following steps:

In the first step, an alumina support comprising an inner core of alpha alumina and an outer layer comprising at least one layer of alumina selected from the group consisting of gamma alumina, delta alumina and theta alumina is prepared.

In the second step, the alumina support is impregnated with at least one alkaline earth metal compound and then dried and calcined at a temperature of 500° C. to 700° C. for a time period ranging between 1 to 10 hours to obtained an alumina support impregnated with at least one alkaline earth metal element.

The alkaline earth metal may be at least one selected from the group consisting of magnesium, calcium, barium and strontium and the alkaline earth metal compound is at least one selected from the group consisting of magnesium nitrate, magnesium acetate, calcium nitrate, barium nitrate and strontium nitrate.

The alkaline earth metal may be magnesium and the amount of magnesium may be maintained in the range of 1 to 10% with respect to the mass of the dehydrogenation catalyst composite of the present disclosure.

In the third step, the alumina support impregnated with at least one alkaline earth metal element is further impregnated with a mixture comprising at least one catalytic metal element compound, at least one group VIA element compound and optionally, at least one halogen element compound to obtain a catalyst composite. The catalyst composite so obtained is then dried and calcined to obtain a calcined catalyst composite impregnated with at least one layer of catalytic metal element and at least one group VIA element.

The catalytic metal element compounds include VIII element compounds, group IVA element compounds, group VIA element compounds, alkali metal element compounds and halogen element compounds in amounts in the range of 0.01 to 5%, 0.01 to 15%, 0.01 to and 2%, 0.01 to 2% respectively with respect to the total mass of the dehydrogenation catalyst composite.

The group VIII element may be at least one selected from the group consisting of platinum, nickel, and palladium and the group VIII element compound may be at least one selected from the group consisting of chloroplatinic acid, palladium nitrate and nickel nitrate.

The group WA element may be at least one selected from the group consisting of tin, and germanium and the group IVA element compound may be at least one selected from the group consisting of stannous chloride and germanium chloride.

The alkali metal elements may be at least one selected from the group consisting of sodium, lithium, potassium and cesium and the alkali metal compound may be at least one selected from the group consisting of sodium chloride, lithium nitrate, potassium chloride and cesium nitrate.

The Group VIA element may be at least one selected form the group consisting of sulfur, selenium and tellurium.

The Group VIA element compound may be at least one selected from the group consisting of thiomalic acid, thioglycolic acid, selenium sulfide and tellurium tetrachloride.

In accordance with one embodiment of the present disclosure the group VIA element compound is thiomalic acid and on calcination thiomalic acid reduces to elemental sulfur.

The halogen element may be at least one selected from the group consisting of chlorine, bromine, fluorine and iodine and the halogen element compound may be at least one selected from the group consisting of hydrochloric acid, carbon tetrachloride, hydrogen bromide, hydrogen fluoride and hydrogen iodide.

In the fourth step, the catalyst composite is contacted with a stream of hydrogen gas under reducing conditions and at a temperature of 400° C. to 500° C. for a time period of 4 to 8 hrs to obtain a dehydrogenation catalyst composite of the present disclosure.

In accordance with one of the embodiments the dehydrogenation catalyst composite of the present disclosure is further blanketed by first purging the dehydrogenation catalyst composite with a stream of inert gas at a temperature in the range of 300° C. to 500° C. and at a gas hourly space velocity (GHSV) of 100 to 10000 and then subsequently cooling the stream to obtain a blanketed dehydrogenation catalyst composite. The gas hourly space velocity (GHSV) of inert gas may be maintained in the range of 100 to 10000.

The alumina support comprising a core of alpha alumina may be prepared by first coating the core with a mixture comprising at least one binder and activated alumina to obtain a coated core.

In one embodiment, the binder is a polar solvent, at least one selected from the group consisting of water, alcohol and ester.

In accordance with one embodiment of the present disclosure the binder is water.

In accordance with one embodiment of the present disclosure binder is provided as a discrete layer between the core and the layer of alumina surrounding the core.

In the next step, the coated core so obtained is hydrated to obtain a hydrated core and then further dried and calcined at a temperature ranging between 800° C. and 900° C. using air to obtain an alumina support having at least one layer comprising at least one alumina selected from the group consisting of gamma alumina, delta alumina and theta alumina.

In accordance with yet another aspect of the present disclosure there is provided a process for preparation of unsaturated hydrocarbons; said process comprising the following steps:
c) preparing a dehydrogenation catalyst composite as the per the process of the present disclosure and
d) contacting said dehydrogenation catalyst composite with hydrocarbon feed at a temperature ranging between 400° C. and 800° C., at a pressure ranging between 0.1 and 10 atm. and at a liquid hourly space velocity (LHSV) in the range of 0.1 to 100 to obtain unsaturated hydrocarbons.

The hydrocarbon feed may comprise at least one hydrocarbon with carbon chain containing C2-C20 atom selected from the group consisting of straight chain paraffins, branched chain paraffins, cyclo-paraffin and a mixture thereof.

Hydrocarbon feed typically may be n-nonane, n-decane, n-dodecane, tridecane and tertadecane.

The present disclosure will now be elaborated in the light of the following non-limiting examples

### Example 1

### Preparation of Alumina Support

Inert alpha alumina spheres of avg. 1.2 mm diameter were used as a core. The core was grown further by coating with an activated alumina powder and a binder in a rotating pan till the core attained an avg. 1.8 mm diameter size. The coated core was then hydrated and subsequently heated at 850° C. temperature in the presence of air. The activated alumina upon heating at 850° C., gave a phase mixture of delta and theta alumina.

### Example-2

### Preparation of a Dehydrogenation Catalyst Composite of the Present Disclosure (Catalyst B)

Employing the two-step impregnation of spheroidal coated alumina support, as prepared in example 1, a catalyst composite was prepared by adopting the incipient wetness technique:

In the first step of impregnation, a solution of MgNO₃ was employed to impregnate the support by wet impregnation. Thereafter the support thus impregnated was dried and calcined at 640° C./4 h. The second impregnation was carried out with the salt solutions of Pt, Sn, and Na. The compounds used were H₂PtCl₆, SnCl₂, NaCl, HCl and TMA. The re-impregnated support was once again dried and calcined.

The wt % of the different elements in the catalyst B are given in table 1

TABLE 1

| | Pt | Sn | Na | Mg | Cl | TMA |
|---|---|---|---|---|---|---|
| | (wt %) | (wt %) | (wt %) | (wt %) | (wt %) | (wt %) |
| Catalyst B | 0.17 | 0.20 | 0.30 | 0.50 | 0.3 | 0.05 |

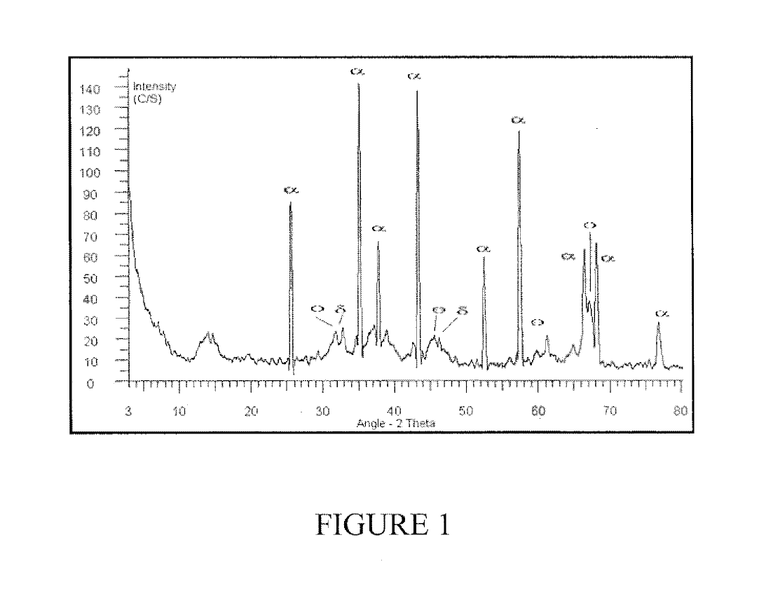

The XRD pattern of dehydrogenation catalyst as illustrated in FIG. 1 shows major peaks, at 2θ: 25.5°, 31.7°, 32.8°, 35.1°, 37.7 °, 43.3°, 45.1°, 46.2°, 52.5°, 57.4°, 61.2°, 66.5°, 67.2°, 68.1°, 76.8° corresponding to alumina phases.

### Example 3

### Effect of Alkaline Earth Metal on Bromine Number of Dehydrogenation Catalyst Composite

Bromine number for the catalyst as prepared in accordance with example 1 and 2 was determined. The comparative bromine numbers of these catalysts are provided in Table 2. It was found that the catalyst of the present disclosure i.e. catalyst B* showed a better bromine number stability compared to catalyst A*.

TABLE 2

| | Bromine Number | Bromine Number | Drop in Bromine |
|---|---|---|---|
| | (Activity) | (Activity) | Number, |
| Catalyst | First Hour | Fifth Hour | stability |
| Catalyst A | 23.0 | 17.5 | 5.5 |
| Catalyst B | 23.5 | 20 | 3.5 |

*Catalyst A-a catalyst comprising a group VIII element platinum as activator, modifier elements tin, iridium and combination of sodium and lithium as promoter elements and also comprising chloride compounds and a group VIA element (capping agent) as thiomalic acid.
*Catalyst B-Catalyst of the present disclosure comprising magnesium in place of lithium and eliminating iridium from Catalyst A.

### Example 4

### Effect of Alkaline Earth Metal on Conversion of n-Paraffin

Conversion of n-dodecane to dodcene for the catalyst as prepared in accordance with example 2 was determined using HPLC. The comparative HPLC conversion of catalyst A & B is provided in Table 3. It was found that catalyst B of the present disclosure shows good conversion and better stability as compared to catalyst A after 7 hours on stream.

TABLE 3

| | Catalyst A | Catalyst B |
|---|---|---|
| Hours | (%) | (%) |
| 1 | 30.29 | 31.26 |
| 2 | 28.74 | 29.79 |
| 3 | 26.73 | 28.59 |
| 4 | 25.07 | 27.99 |
| 5 | 23.39 | 26.78 |
| 6 | 22.13 | 25.32 |
| 7 | 20.17 | 25.66 |

The deactivation percentage for these catalysts after 7 hours is provided in Table 4. It was found that catalyst B of the present disclosure shows lower deactivation percentage (19%) than catalyst A (33%). Due to the lower catalyst deactivation percentage, the stability of catalyst B is 42% higher than that of catalyst A.

The deactivation percentage is calculated by D=[(Initial activity−activity (t))/Initial activity]×100

TABLE 4

| Catalyst | Deactivation Percentage (D), % |
|---|---|
| Catalyst A | 33 |
| Catalyst of the present | 19.0 |
| disclosure | |

### Example 5

### Effect of Alkaline Earth Metal on the Selectivity of Mon-Olefins and Aromatics

The comparative HPLC analysis in order to detect the selectivities of catalyst A and catalyst B for the n-decane dehydrogenation under similar reaction condition is provided in Table 5. It was found that catalyst B of the present disclosure shows 1.8% higher mono-olefin desired selectivity than catalyst B. It was also observed that, catalyst B shows 33% lower aromatics formation than catalyst A during the dehydrogenation process, which is responsible for coke formation and catalyst deactivation. Due to lower aromatics formation, the stability and life of catalyst B is higher than that of catalyst A.

TABLE 5

| | N-Decane | Mono-Olefin | Di-Olefin | Aromatics, |
|---|---|---|---|---|
| | Conversion, | Selectivity, | Selectivity, | Selectivity, |
| Catalyst | % | % | % | % |
| Catalyst A | 12.7 | 91.0 | 4.8 | 4.2 |
| Catalyst B | 12.6 | 92.7 | 4.5 | 2.8 |

### Example 6

### Effect of Alkaline Earth Metal on Dispersion of Active Catalyst

Hydrogen chemisorption method was used for the determination of dispersion and average crystallite size of the platinum particles (Active catalyst) supported on alumina in catalyst A and catalyst B. The monolayer uptake, metal dispersion and average crystallite size of platinum particles in catalyst A and catalyst B are given in the following Table 6.

TABLE 6

| | | | Monolayer | |
|---|---|---|---|---|
| | | Metal | Uptake, | Average |
| | H₂:Pt | Dispersion, | μmol/g | Crystallite |
| | Stiocho- | % at | (moles of H₂/ | Size, |
| Catalyst | metric | 150° C. | gm of Pt) | nm |
| Catalyst | 2 | 46 | 2.02 | 2.4 |
| A* | | | | |
| Catalyst | 2 | 62 | 2.65 | 1.8 |
| B* | | | | |

It was observed that, the monolayer uptake is higher in catalyst B (2.65 μmol/g) over catalyst A (2.02 μmol/g) which corresponds to more number of platinum active sites available for dehydrogenation. The average crystallite size of the platinum metal in catalyst A (1.8 nm) is lower than that in catalyst B (2.4 nm).

The Pt dispersion in catalyst A was determined as 46% by H2 chemisorption method; whereas in catalyst B, Pt metal dispersion was 62%. In catalyst B, the number of active Pt sites available on the surface are higher which corresponds to good activity, selectivity and stability for dehydrogenation reactions.

The embodiments herein and the various features and advantageous details thereof are explained with reference to the non-limiting embodiments in the description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

### Technical Advantages and Economic Significance

The dehydrogenation catalyst composite prepared in accordance with the present disclosure has improved stability and better dispersion of the catalytic metal elements.

Further, the dehydrogenation catalyst composite prepared in accordance with the present disclosure is safe and economic.

Still further the alkaline earth metal used in the dehydrogenation catalyst composite of the present disclosure improves the stability of the catalyst.

Even further, the process of the present disclosure obviates the use of costly catalyst such as iridium, thereby making the dehydrogenation catalyst composite more cost effective.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The use of the expression “a”, “at least” or “at least one” suggests the use of one or more elements or ingredients or quantities, as the use may be in the embodiment of the disclosure to achieve one or more of the desired objects or results.

The numerical values given for various physical parameters, dimensions and quantities are only approximate values and it is envisaged that the values higher or lower than the numerical value assigned to the physical parameters, dimensions and quantities fall within the scope of the disclosure and the claims unless there is a statement in the specification to the contrary.

While certain embodiments of the disclosure have been described, these embodiments have been presented by way of examples only, and are not intended to limit the scope of the disclosure. Variations or modifications in the process of this disclosure, within the scope of the disclosure, may occur to those skilled in the art upon reviewing the disclosure herein. Such variations or modifications are well within the spirit of this disclosure.

1. A dehydrogenation catalyst composite comprising:
a. at least one alumina support comprising:
i. a core of alpha alumina; and
ii. at least one layer of alumina selected from the group consisting of gamma alumina, delta alumina and theta alumina surrounding said core,
b. at least one layer comprising at least one alkaline earth metal element selected from the group consisting of magnesium, calcium, barium and strontium impregnated on the surface of said alumina support; and
c. at least one layer comprising:
i. at least one catalytic metal element selected from the group consisting of group VIII elements, group IVA elements, and alkali metal elements;
ii. at least one group VIA element; and
iii. optionally, at least one halogen element,
said layer provided on alkaline earth metal impregnated alumina support.

2. The dehydrogenation catalyst composite of claim 1, characterized in that the percentage dispersion of catalytic metal element is in the range of 55% to 80%.

3. The catalyst composite as claimed in claim 1, which further comprises at least one binder provided within at least one layer of alumina and/or as a discrete layer between the core and the layer of alumina surrounding the core.

4. The catalyst composite as claimed in claim 2, wherein the binder is at least one polar compound selected from the group consisting of water, alcohol and ester, preferably water.

5. The catalyst composite as claimed in claim 1, wherein the average diameter of the alumina support is in the range of 1.8 mm to 2.00 mm and the surface area is in the range of 10 m²/g to 200 m²/g.

6. The catalyst composite as claimed in claim 1, wherein the amount of alkaline earth metal element impregnated on the alumina support is in the range of 1% to 10% with respect to the total mass of the dehydrogenation catalyst composite.

7. The catalyst composite as claimed in claim 1, wherein the group VIII element is at least one selected from the group consisting of platinum, nickel and palladium, the group IVA element is at least one selected from the group consisting of tin and germanium, the alkali metal element is at least one selected from the group consisting of sodium, lithium, potassium and cesium, the halogen element is at least one selected from the group consisting of chlorine, bromine, fluorine and iodine, the group VIA element is at least one selected from the group consisting of sulfur, selenium and tellurium, preferably sulfur and the amount of VIA element ranges between 0.01% and 15% with respect to the total mass of the dehydrogenation catalyst composite.

8. (canceled)

9. (canceled)

10. (canceled)

11. The catalyst composite as claimed in claim 1, wherein the amount of group VIII elements ranges between 0.01 and 5%, the amount of group IVA elements ranges between 0.01 and 15%, the amount of alkali metal element ranges between 0.01 and 2% and the amount of halogen element ranges between 0.05 and 0.5%; wherein said amount of each element is based on the total mass of the dehydrogenation catalyst composite.

12. (canceled)

13. (canceled)

14. A process for the preparing a dehydrogenation catalyst composite, said process comprising the following steps:
a. preparing an alumina support; said method step of preparing an alumina support comprises the following steps:
I. obtaining a core of alpha alumina;
II. coating the core with a mixture comprising activated alumina and at least one binder to obtain a coated core;
III. hydrating the coated core to obtain hydrated core; and
IV. calcining the hydrated core at a temperature of 800 to 900° C. in presence of air to obtain an alumina support with at least one layer of at least one alumina selected from the group consisting of gamma alumina, delta alumina and theta alumina,
b. impregnating the alumina support with at least one alkaline earth metal element compound followed by drying and calcining at a temperature of 500° C. to 700° C. for a time period ranging between 1 to 10 hours to obtain an alumina support impregnated with at least one alkaline earth metal element;
c. impregnating the alumina support impregnated with at least one alkaline earth metal element with a mixture comprising at least one catalytic metal element compound, at least one group VIA element compound and optionally, at least one halogen element compound to obtain a catalyst composite; wherein the catalytic metal element compounds is at least one selected from the group consisting of group VIII element compounds, group IVA element precursors and alkali metal element compounds;
d. drying and calcining the catalyst composite to obtain a calcined catalyst composite impregnated with at least one catalytic metal element and at least group VIA element; and
e. contacting the calcined catalyst composite with a stream of hydrogen gas under reducing conditions to obtain a dehydrogenation catalyst composite.

15. The process as claimed in claim 14, wherein the binder is at least one polar solvent selected from the group consisting of water, alcohol and ester, preferably water.

16. The process as claimed in claim 14, further comprises the following steps:
a) purging a stream of inert gas at a temperature of 300° C. to 500° C. at a high gas hourly space velocity of 100 to 10000 per hour on the dehydrogenation catalyst composite; and
b) cooling the stream to obtain a blanketed dehydrogenation catalyst composite.

17. (canceled)

18. (canceled)

19. (canceled)

20. (canceled)

21. (canceled)

22. The process as claimed in claim 14, wherein the group VIII element compound is at least one selected from the group consisting of chloroplatinic acid, palladium nitrate and nickel nitrate.

23. (canceled)

24. The process as claimed in claim 14, wherein the group IVA element compound is at least one selected from the group consisting of stannous chloride and germanium chloride.

25. (canceled)

26. The process as claimed in claim 14, wherein the alkali metal compound is at least one selected from the group consisting of sodium chloride, lithium nitrate, potassium chloride and cesium nitrate.

27. (canceled)

28. (canceled)

29. (canceled)

30. (canceled)

31. (canceled)

32. The process as claimed in claim 14, wherein the hydrogen gas is maintained at a temperature of 400 to 500° C. for a time period of 4 to 8 hrs.

33. A process for the preparation of unsaturated hydrocarbons; said process comprising the following steps:
a) preparing a dehydrogenation catalyst composite by the process as claimed in claim 14; and
b) contacting said dehydrogenation catalyst composite with at least one hydrocarbon feed at a temperature ranging between 400° C. and 800° C., at a pressure ranging between 0.1 and 10 atm and at a liquid hourly space velocity in the range of 0.1 to 100/hr to obtain unsaturated hydrocarbons.

34. The process as claimed in claim 33, wherein the hydrocarbon feed comprises at least one hydrocarbon selected from the group consisting of C2 to C20 hydrocarbons.
